(19) United States
(12) Patent Application Publication
(10) Pub. No.: US 2009/0318565 A1
(43) Pub. Date: Dec. 24, 2009

(54) Novel Compositions for the Enhancement of the Anti-oxidant Potential of Hydrogenated Curcuminoids or Combinations Thereof

(76) Inventor: Muhammed Majeed, Piscataway, NJ (US)

Correspondence Address: SABINSA CORPORATION, 70 ETHEL ROAD WEST, UNIT 6, PISCATAWAY, NJ, 08854, US

(21) Appl. No.: 12/142,937

(22) Filed: Jun. 20, 2008

(51) Int. Cl. A61K 31/12; A61K 31/085; A61P 43/00

(52) U.S. Cl. 514/679; 514/721

(57) ABSTRACT

Disclosed are novel compositions for the enhancement of the anti-oxidant potential of natural or metabolite hydrogenated (1E,6E)-1,7-Bis(4-hydroxy-3-methoxyphenyl)-1,6-heptadiene-3,5-dione derivatives. It was discovered that the antioxidant potential of hydrogenated curcuminoids was highly enhanced when a closely structurally related 1,7-Bis(4-hydroxyphenyl)-3,5-heptanedione, exhibiting unexpectedly pro-oxidant properties, was removed from the compositions.

### BACKGROUND OF THE INVENTION

1. Field of the Invention

The present invention in general relates to natural and metabolite derivatives of hydrogenated (1E,6E)-1,7-Bis (4-hydroxy-3-methoxyphenyl)-1,6-heptadiene-3,5-Dione. More specifically, the present invention relates to compositions for the enhancement of the anti-oxidant potential of the natural or metabolite hydrogenated (1E,6E)-1,7-Bis (4-hydroxy-3-methoxyphenyl)-1,6-heptadiene-3,5-dione derivatives or combinations thereof, said compositions occurring devoid of the co-occurring pro-oxidant 1,7-Bis(4-hydroxyphenyl)-3,5-heptanedione.

2. Description of Prior Art

The inventors of the present invention have observed that upon hydrogenation of naturally occurring curcumin ((1E,6E)-1,7-Bis(4-hydroxy-3-methoxyphenyl)-1,6-heptadiene-3,5-dione), the anti-oxidant properties of its natural demethoxyderivative, demethoxycurcumin (DMC)] hydrogenated as 1-(4-hydroxy-3-methoxyphenyl)-7-(4-hydroxyphenyl)-3,5-heptanedione (tetrahydrodemethoxycurcumin) is enhanced about 10 fold as demonstrated by the Reactive Oxygen Species Scavenging (ROS) activity. Similarly, the anti-oxidant activity of hydrogenated metabolite derivative of curcumin,1,7-Bis(4-hydroxy-3-methoxyphenyl)-3,5-heptanedione (tetrahydrocurcumin) is also enhanced. The pro-oxidant properties of bisdemethoxycurcumin have already been reported in the research publication titled “Pro-oxidant, anti-oxidant and cleavage activities on DNA of curcumin and its derivatives demethoxycurcumin and bisdemethoxycurcumin” in Chem Biol Interact. Jul. 1, 1999; 121(2):161-75 by Ahsan H, Parveen N, Khan N U, Hadi S M, Department of Biochemistry, Faculty of Life Sciences, Aligarh Muslim University, India. The inventors of the present invention have also found that upon hydrogenation of naturally occurring curcumin (1E,6E)-1,7-Bis (4-hydroxy-3-methoxyphenyl)-1,6-heptadiene-3,5-dione), the pro-oxidant properties of its natural demethoxyderivative, bisdemethoxycurcumin hydrogenated as 1,7-Bis(4-hydroxyphenyl)-3,5-heptanedione(tetrahydrobisdemethoxycurcumin) is also enhanced. Such an enhanced pro-oxidant effect brings down the anti-oxidant potential of other natural or metabolite hydrogenated derivatives of 1E,6E)-1,7-Bis (4-hydroxy-3-methoxyphenyl)-1,6-heptadiene-3,5-dione and anti-oxidant compositions thereof. Hence, the inventors of the present invention have proposed compositions for the enhancement of the anti-oxidant potential of natural and metabolite hydrogenated derivatives of 1E,6E)-1,7-Bis(4-hydroxy-3-methoxyphenyl)-1,6-heptadiene-3,5-dione or combinations thereof, said compositions occurring devoid of the pro-oxidant 1,7-Bis(4-hydroxyphenyl)-3,5-heptanedione.

Accordingly, it is the principle object of the present invention to provide anti-oxidant compositions comprising natural or metabolite hydrogenated (1E,6E)-1,7-Bis(4-hydroxy-3-methoxyphenyl)-1,6-heptadiene-3,5-dione derivatives or combinations thereof, said compositions occurring devoid of co-occurring pro-oxidant 1,7-Bis(4-hydroxyphenyl)-3,5-heptanedione.

The present invention fulfills the aforementioned needs and provides further related advantages.

### SUMMARY OF THE INVENTION

The present invention relates to novel compositions for the enhancement of the anti-oxidant potential of natural or metabolite hydrogenated (1E,6E)-1,7-Bis (4-hydroxy-3-methoxyphenyl)-1,6-heptadiene-3,5-dione derivatives or combinations thereof, said compositions occurring devoid of the co-occurring pro-oxidant 1,7-Bis(4-hydroxyphenyl)-3,5-heptanedione.

Other features and advantages of the present invention will become apparent from the following more detailed description, taken in conjunction with the accompanying drawings, which illustrate, by way of example, the principle of the invention.

### BRIEF DESCRIPTION OF THE DRAWINGS

FIG. 1 shows the graphical representation of anti-oxidant profiles of (1E,6E)-1,7-Bis(4-hydroxy-3-methoxyphenyl)-1,6-heptadiene-3,5-dione, its natural and metabolite hydrogenated derivatives in ROS scavenging assay performed using Swiss 3T3 fibroblast cell line.

### DESCRIPTION OF THE MOST PREFERRED EMBODIMENT

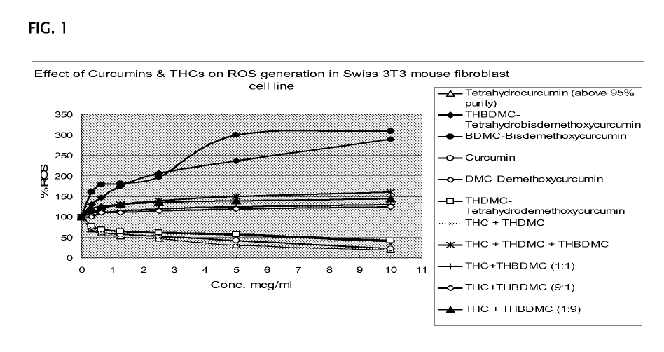

### FIG. 1, Table 1

In the most preferred embodiment, the present invention relates to novel compositions for the enhancement of the anti-oxidant potential of the natural or metabolite hydrogenated (1E,6E)-1,7-Bis (4-hydroxy-3-methoxyphenyl)-1,6-heptadiene-3,5-dione derivatives or combinations thereof, said compositions occurring devoid of the co-occurring pro-oxidant 1,7-Bis(4-hydroxyphenyl)-3,5-heptanedione represented by STR#1.

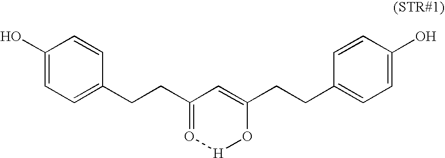

More preferably, the present invention discloses anti-oxidant compositions comprising
a) 99.5% pure 1,7-Bis(4-hydroxy-3-methoxyphenyl)-3,5-heptanedione represented by STR#II, not occurring along with 5-Hydroxy-1,7-bis(4-hydroxy-3-methoxyphenyl)-3-heptanone represented by STR#III or 3,5-Dihydroxy-1,7-bis(4-hydroxy-3-methoxyphenyl)-heptane represented by STR#IV and occurring devoid of pro-oxidant 1,7-Bis(4-hydroxyphenyl)-3,5-heptanedione represented by STR#1;
b) 99.5% pure 1,7-Bis(4-hydroxy-3-methoxyphenyl)-3,5-heptanedione represented by STR#II, not occurring along with 5-Hydroxy-1,7-bis(4-hydroxy-3-methoxyphenyl)-3-heptanone represented by STR#III or 3,5-Dihydroxy-1,7-bis(4-hydroxy-3-methoxyphenyl)-heptane represented by STR#IV, in combination with 1-(4-hydroxy-3-methoxyphenyl)-7-(4-hydroxyphenyl)-3,5-heptanedione represented by STR#V, and said combination occurring devoid of pro-oxidant 1,7-Bis(4-hydroxyphenyl)-3,5-heptanedione represented by STR#1;
c) 1 -(4-hydroxy-3-methoxyphenyl)-7-(4-hydroxyphenyl)-3,5-heptanedione represented by STR#V, occurring devoid of pro-oxidant 1,7-Bis(4-hydroxyphenyl)-3,5-heptanedione represented by STR#1;
d) 5-Hydroxy-1,7-bis(4-hydroxy-3-methoxyphenyl)-3-heptanone represented by STR#III, occurring devoid of pro-oxidant 1,7-Bis(4-hydroxyphenyl)-3,5-heptanedione represented by STR#1; and
e) 3,5-Dihydroxy-1,7-bis(4-hydroxy-3-methoxyphenyl)-heptane represented by STR#IV, occurring devoid of pro-oxidant 1,7-Bis(4-hydroxyphenyl)-3,5-heptanedione represented by STR#1.

Table 1 (EXAMPLE I) shows the anti-oxidant profiles of curcumin, its natural derivatives and its metabolite hydrogenated derivatives or combinations thereof in the 1,1-diphenyl-2-picryl hydrazyl radical (DPPH) assay and the ROS scavenging assay. The DPPH free radical scavenging activity is studied based on the stable free radical 1,1-diphenyl-2-picryl hydrazyl radical (DPPH) which, in presence of an antioxidant in organic/aqueous organic media is bleached and the decrease of absorbance of DPPH is measured spectrophotometrically at 516 nm. The assay mixture containing DPPH methanolic solution & methanolic solutions of various concentrations of the antioxidant is incubated at 37 degree C. for 30 minutes. The reduction in absorbance is measured spectrophotometrically at 516 nm. The free radical scavenging activity is expressed as SC₅₀, the concentration of the sample required for 50% of the free radical to be scavenged. The ROS scavenging assay is based on a cell permeable, non-fluorescent dye, de-esterified DCFH-DA (dichloro fluorescein diacetate) being converted to dichlorofluorescein upon oxidation by the reactive oxygen species. The scavenging activity of sample is indicated by the decrease in fluorescence (wavelength 485/520 nm). Swiss 3T3 mouse fibroblast cells are cultured in DMEM medium with 10% FBS. 1×10⁵ cells per well are plated into a 96 well black plate. After treatment with varying concentrations of the samples, the cells are exposed to DCFH-DA dye and oxidation initiated by FeSO4. After incubation for 1 hr at 37° C., the fluorescence reading is taken at wavelength 485/520 nm (Fluostar optima microplate reader). The fluorescence intensity is directly proportional to the ROS scavenging activity of the samples. The activity is expressed as SC₅₀ which is the concentration required for 50% scavenging of ROS.

TABLE 1

| | % Inhibition (IC50 μg/ml) | |
|---|---|---|
| | DPPH free | |
| | radical | ROS scavenging in Swiss |
| Sample | scavenging | 3T3 fibroblast cell line |
| 99.5% pure 1,7-Bis(4-hydroxy-3- | 1.2 | 1.35 |
| methoxyphenyl)-3,5-heptanedione | | |
| represented by STR#II, not occurring | | |
| along with 5-Hydroxy-1,7-bis(4- | | |
| hydroxy-3-methoxyphenyl)-3- | | |
| heptanone represented by STR#III or | | |
| 3,5-Dihydroxy-1,7-bis(4-hydroxy-3- | | |
| methoxyphenyl)-heptane represented | | |
| by STR#IV, said composition being | | |
| devoid of pro-oxidant 1,7-Bis(4- | | |
| hydroxyphenyl)- 3,5-heptanedione | | |
| represented by STR#1; | | |
| 1-(4-hydroxy-3-methoxyphenyl)-7- | 3.0 | 4.5 |
| (4-hydroxyphenyl)-3,5-heptanedione | | |
| (STR#V) and said composition being | | |
| devoid of pro-oxidant 1,7-Bis(4- | | |
| hydroxyphenyl)- 3,5-heptanedione | | |
| represented by STR#1 | | |
| 99.5% pure 1,7-Bis(4-hydroxy-3- | 2.08 | 2.5 |
| methoxyphenyl)-3,5-heptanedione | | |
| represented by STR#II, not occurring | | |
| along with 5-Hydroxy-1,7-bis(4- | | |
| hydroxy-3-methoxyphenyl)-3- | | |
| heptanone represented by STR#III or | | |
| 3,5-Dihydroxy-1,7-bis(4-hydroxy-3- | | |
| methoxyphenyl)-heptane represented | | |
| by STR#IV, in combination with 1-(4- | | |
| hydroxy-3-methoxyphenyl)-7-(4- | | |
| hydroxyphenyl)-3,5-heptanedione | | |
| represented by STR#V, and said | | |
| composition being devoid of pro- | | |
| oxidant 1,7-Bis(4-hydroxyphenyl)- | | |
| 3,5-heptanedione represented by | | |
| STR#1 (1:1) | | |
| 99.5% pure 1,7-Bis(4-hydroxy-3- | 2.5 | 3.0 |
| methoxyphenyl)-3,5-heptanedione | | |
| represented by STR#II, not occurring | | |
| along with 5-Hydroxy-1,7-bis(4- | | |
| hydroxy-3-methoxyphenyl)-3- | | |
| heptanone represented by STR#III or | | |
| 3,5-Dihydroxy-1,7-bis(4-hydroxy-3- | | |
| methoxyphenyl)-heptane represented | | |
| by STR#IV, in combination with 1-(4- | | |
| hydroxy-3-methoxyphenyl)-7-(4- | | |
| hydroxyphenyl)-3,5-heptanedione | | |
| represented by STR#V, and said | | |
| composition being devoid of pro- | | |
| oxidant 1,7-Bis(4-hydroxyphenyl)- | | |
| 3,5-heptanedione represented by | | |
| STR#1 (1:9) | | |
| 99.5% pure 1,7-Bis(4-hydroxy-3- | 1.4 | 2.0 |
| methoxyphenyl)-3,5-heptanedione | | |
| represented by STR#II, not occurring | | |
| along with 5-Hydroxy-1,7-bis(4- | | |
| hydroxy-3-methoxyphenyl)-3- | | |
| heptanone represented by STR#III or | | |
| 3,5-Dihydroxy-1,7-bis(4-hydroxy-3- | | |
| methoxyphenyl)-heptane represented | | |
| by STR#IV, in combination with 1-(4- | | |
| hydroxy-3-methoxyphenyl)-7-(4- | | |
| hydroxyphenyl)-3,5-heptanedione | | |
| represented by STR#V, and said | | |
| composition being devoid of pro- | | |
| oxidant 1,7-Bis(4-hydroxyphenyl)- | | |
| 3,5-heptanedione represented by | | |
| STR#1 (9:1) | | |
| 99.5% pure 1,7-Bis(4-hydroxy-3- | 1.5 | 10 |
| methoxyphenyl)-3,5-heptanedione | | |
| represented by STR#II, not occurring | | |
| along with 5-Hydroxy-1,7-bis(4- | | |
| hydroxy-3-methoxyphenyl)-3- | | |
| heptanone represented by STR#III or | | |
| 3,5-Dihydroxy-1,7-bis(4-hydroxy-3- | | |
| methoxyphenyl)-heptane represented | | |
| by STR#IV, in combination with 1-(4- | | |
| hydroxy-3-methoxyphenyl)-7-(4- | | |
| hydroxyphenyl)-3,5-heptanedione | | |
| represented by STR#V, and pro-oxidant | | |
| 1,7-Bis(4-hydroxyphenyl)- 3,5- | | |
| heptanedione represented by | | |
| STR#1 (9:1:1) | | |
| 99.5% pure 1,7-Bis(4-hydroxy-3- | 20 | No ROS scavenging activity |
| methoxyphenyl)-3,5-heptanedione | | |
| represented by STR#II, not occurring | | |
| along with 5-Hydroxy-1,7-bis(4- | | |
| hydroxy-3-methoxyphenyl)-3- | | |
| heptanone represented by STR#III or | | |
| 3,5-Dihydroxy-1,7-bis(4-hydroxy-3- | | |
| methoxyphenyl)-heptane represented | | |
| by STR#IV, in combination with 1-(4- | | |
| hydroxy-3-methoxyphenyl)-7-(4- | | |
| hydroxyphenyl)-3,5-heptanedione | | |
| represented by STR#V, and pro-oxidant | | |
| 1,7-Bis(4-hydroxyphenyl)- 3,5- | | |
| heptanedione represented by STR#1 | | |
| (1:1:9) | | |
| 99.5% pure 1,7-Bis(4-hydroxy-3- | 3.0 | 10 |
| methoxyphenyl)-3,5-heptanedione | | |
| represented by STR#II, not occurring | | |
| along with 5-Hydroxy-1,7-bis(4- | | |
| hydroxy-3-methoxyphenyl)-3- | | |
| heptanone represented by STR#III or | | |
| 3,5-Dihydroxy-1,7-bis(4-hydroxy-3- | | |
| methoxyphenyl)-heptane represented | | |
| by STR#IV, in combination with 1-(4- | | |
| hydroxy-3-methoxyphenyl)-7-(4- | | |
| hydroxyphenyl)-3,5-heptanedione | | |
| represented by STR#V, and pro-oxidant | | |
| 1,7-Bis(4-hydroxyphenyl)- 3,5- | | |
| heptanedione represented by STR#1 | | |
| (1:1:1) | | |
| 99.5% pure 1,7-Bis(4-hydroxy-3- | 8.0 | Decrease in ROS |
| methoxyphenyl)-3,5-heptanedione | | scavenging activity of THC |
| represented by STR#II, not occurring | | by 10 times |
| along with 5-Hydroxy-1,7-bis(4- | | (IC₅₀ of THC shifted from |
| hydroxy-3-methoxyphenyl)-3- | | the value of 1 to 10) |
| heptanone represented by STR#III or | | |
| 3,5-Dihydroxy-1,7-bis(4-hydroxy-3- | | |
| methoxyphenyl)-heptane represented | | |
| by STR#IV in combination with pro- | | |
| oxidant 1,7-Bis(4-hydroxyphenyl)- | | |
| 3,5-heptanedione represented by | | |
| STR#1 (1:1) | | |
| 99.5% pure 1,7-Bis(4-hydroxy-3- | 2.8 | Decrease in ROS |
| methoxyphenyl)-3,5-heptanedione | | scavenging activity of THC |
| represented by STR#II, not occurring | | by 3 times |
| along with 5- Hydroxy-1,7-bis(4- | | (IC₅₀ of THC shifted from |
| hydroxy-3-methoxyphenyl)-3- | | the vlaue of 1 to 3) |
| heptanone represented by STR#III or | | |
| 3,5-Dihydroxy-1,7-bis(4-hydroxy-3- | | |
| methoxyphenyl)-heptane represented | | |
| by STR#IV in combination with pro- | | |
| oxidant 1,7-Bis(4-hydroxyphenyl)- | | |
| 3,5-heptanedione represented by | | |
| STR#1 (9:1) | | |
| 99.5% pure 1,7-Bis(4-hydroxy-3- | 18.6 | No ROS scavenging activity |
| methoxyphenyl)-3,5-heptanedione | | |
| represented by STR#II, not occurring | | |
| along with 5-Hydroxy-1,7-bis(4- | | |
| hydroxy-3-methoxyphenyl)-3- | | |
| heptanone represented by STR#III or | | |
| 3,5-Dihydroxy-1,7-bis(4-hydroxy-3- | | |
| methoxyphenyl)-heptane represented | | |
| by STR#IV in combination with pro- | | |
| oxidant 1,7-Bis(4-hydroxyphenyl)- | | |
| 3,5-heptanedione represented by | | |
| STR#1 (1:9) | | |
| 5-Hydroxy-1,7-bis(4-hydroxy-3- | 2.6 | Decrease in ROS |
| methoxyphenyl)-3-heptanone | | scavenging activity of THC |
| represented by STR#III in combination | | by 2 times |
| with pro-oxidant 1,7-Bis(4- | | (IC₅₀ of THC shifted from |
| hydroxyphenyl)- 3,5-heptanedione | | the value of 1 to 2) |
| represented by STR#1 (1:1) | | |
| 5-Hydroxy-1,7-bis(4-hydroxy-3- | 1.06 | 12 |
| methoxyphenyl)-3-heptanone | | |
| represented by STR#III in combination | | |
| with pro-oxidant 1,7-Bis(4- | | |
| hydroxyphenyl)- 3,5-heptanedione | | |
| represented by STR#1 (9:1) | | |
| 5-Hydroxy-1,7-bis(4-hydroxy-3- | 15 | 17% increase in ROS |
| methoxyphenyl)-3-heptanone | | generation |
| represented by STR#III in combination | | |
| with pro-oxidant 1,7-Bis(4- | | |
| hydroxyphenyl)- 3,5-heptanedione | | |
| represented by STR#1 (1:9) | | |
| 3,5-Dihydroxy-1,7-bis(4-hydroxy-3- | 2.4 | Decrease in ROS |
| methoxyphenyl)-heptane represented | | scavenging activity of THC |
| by STR#IV in combination with pro- | | by 2 times |
| oxidant 1,7-Bis(4-hydroxyphenyl)- | | (IC₅₀ of THC shifted from |
| 3,5-heptanedione represented by | | the value of 1 to 2) |
| STR#1 (1:1) | | |
| 3,5-Dihydroxy-1,7-bis(4-hydroxy-3- | 1.26 | 15 |
| methoxyphenyl)-heptane represented | | |
| by STR#IV in combination with pro- | | |
| oxidant 1,7-Bis(4-hydroxyphenyl)- | | |
| 3,5-heptanedione represented by | | |
| STR#1 (9:1) | | |
| 3,5-Dihydroxy-1,7-bis(4-hydroxy-3- | 14 | 15% increase in ROS |
| methoxyphenyl)-heptane represented | | generation |
| by STR#IV in combination with pro- | | |
| oxidant 1,7-Bis(4-hydroxyphenyl)- | | |
| 3,5-heptanedione represented by | | |
| STR#1 (1:9) | | |
| 1-(4-hydroxy-3-methoxyphenyl)-7- | 6.45 | 15 |
| (4-hydroxyphenyl)-3,5-heptanedione | | |
| (STR#V) in combination with pro- | | |
| oxidant 1,7-Bis(4-hydroxyphenyl)- | | |
| 3,5-heptanedione represented by | | |
| STR#1 (1:1) | | |
| 1-(4-hydroxy-3-methoxyphenyl)-7- | 3.4 | 10 |
| (4-hydroxyphenyl)-3,5-heptanedione | | |
| (STR#V) in combination with pro- | | |
| oxidant 1,7-Bis(4-hydroxyphenyl)- | | |
| 3,5-heptanedione represented by | | |
| STR#1 (9:1) | | |
| 1-(4-hydroxy-3-methoxyphenyl)-7- | 24 | 20% increase in ROS |
| (4-hydroxyphenyl)-3,5-heptanedione | | generation |
| (STR#V) in combination with pro- | | |
| oxidant 1,7-Bis(4-hydroxyphenyl)- | | |
| 3,5-heptanedione represented by | | |
| STR#1 (1:9) | | |

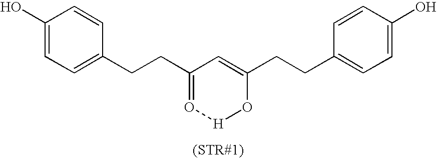

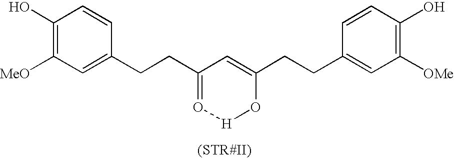

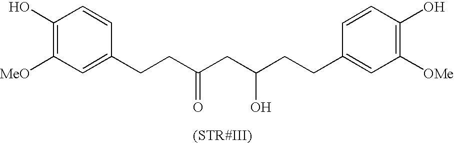

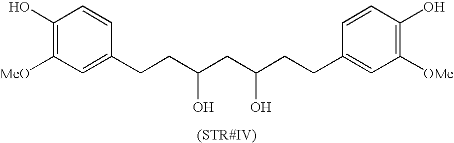

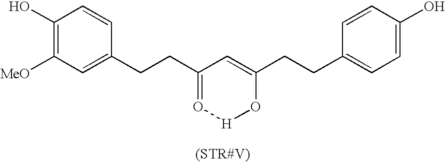

It is clear from the aforesaid table that the presence of very minute quantities of 1,7-Bis (4-hydroxyphenyl)-3,5-heptanedione represented by STR#1 adversely affects the anti-oxidant potential of natural or metabolite hydrogenated (1E,6E)-1,7-Bis (4-hydroxy-3-methoxyphenyl)-1,6-heptadiene-3,5-dione derivatives and combinations thereof. The present inventors have made use of the observation that the pro-oxidant properties of bisdemethoxycurcumin is further enhanced when bisdemethoxy curcumin is hydrogenated as 1,7-Bis (4-hydroxyphenyl)-3,5-heptanedione and the anti-oxidant potential of the natural or metabolite hydrogenated (1E,6E)-1,7-Bis (4-hydroxy-3-methoxyphenyl)-1,6-heptadiene-3,5-dione derivatives or combinations thereof is enhanced when the said derivatives or combinations thereof occur devoid of the pro-oxidant 1,7-Bis (4-hydroxyphenyl)-3,5-heptanedione.

While the invention has been described with reference to a preferred embodiment, it is to be clearly understood by those skilled in the art that the invention is not limited thereto. Rather, the scope of the invention is to be interpreted only in conjunction with the appended claims.

We claim:

1. Antioxidant compositions containing natural or metabolite hydrogenated (1E,6E)-1,7-Bis(4-hydroxy-3-methoxyphenyl)-1,6-heptadiene-3,5-dione derivatives or combinations thereof, the said compositions are devoid of the co-occurring pro-oxidant 1,7-Bis(4-Hydroxyphenyl)-3,5-heptanedione represented by STR#1

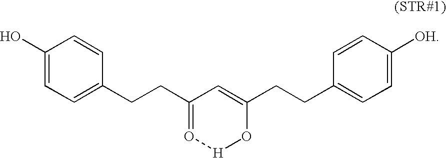

2. The compositions according to claim 1, wherein the said derivatives or combinations thereof include:
a) 99.5% pure 1,7-Bis(4-hydroxy-3-methoxyphenyl)-3,5-heptanedione represented by STR#II, not occurring along with 5-Hydroxy-1,7-bis(4-hydroxy-3-methoxyphenyl)-3-heptanone represented by STR#III or 3,5-Dihydroxy-1,7-bis(4-hydroxy-3-methoxyphenyl)-heptane represented by STR#IV and occurring devoid of pro-oxidant 1,7-Bis(4-hydroxyphenyl)-3,5-heptanedione represented by STR#1;
b) 99.5% pure 1,7-Bis(4-hydroxy-3-methoxyphenyl)-3,5-heptanedione represented by STR#II, not occurring along with 5-Hydroxy-1,7-bis(4-hydroxy-3-methoxyphenyl)-3-heptanone represented by STR#III or 3,5-Dihydroxy-1,7-bis(4-hydroxy-3-methoxyphenyl)-heptane represented by STR#IV, in combination with 1-(4-hydroxy-3-methoxyphenyl)-7-(4-hydroxyphenyl)-3,5-heptanedione represented by STR#V, and said combination occurring devoid of pro-oxidant 1,7-Bis(4-hydroxyphenyl)-3,5-heptanedione represented by STR#1;
c) 1 -(4-hydroxy-3-methoxyphenyl)-7-(4-hydroxyphenyl)-3,5-heptanedione represented by STR#V, occurring devoid of pro-oxidant 1,7-Bis(4-hydroxyphenyl)-3,5-heptanedione represented by STR#1;
d) 5-Hydroxy-1,7-bis(4-hydroxy-3-methoxyphenyl)-3-heptanone represented by STR#III, occurring devoid of pro-oxidant 1,7-Bis(4-hydroxyphenyl)-3,5-heptanedione represented by STR#1; and
e) 3,5-Dihydroxy-1,7-bis(4-hydroxy-3-methoxyphenyl)-heptane represented by STR#IV, occurring devoid of pro-oxidant 1,7-Bis(4-hydroxyphenyl)-3,5-heptanedione represented by STR#1. [chemical structure]
